(19) United States
(12) Patent Application Publication
(10) Pub. No.: US 2003/0044308 A1
(43) Pub. Date: Mar. 6, 2003

(54) Method and article for concentrating and transporting ozone at a desired location

(76) Inventor: Julius Toth, Barberton, OH (US)

Correspondence Address: Roger D. Emerson, One Cascade Plaza, The Fourteenth Floor, Akron, OH, 44308-1136, US

(21) Appl. No.: 09/941,510

(22) Filed: Aug. 28, 2001

(51) Int. Cl. A61L 2/00; A61L 9/00

(52) U.S. Cl. 422/5; 422/28; 422/186.07; 422/186.12

(57) ABSTRACT

A method and articles for concentrating and delivering generated-ozone to specific locations, on a selective and retrofit basis to existing oxygen-generating machines is disclosed. The inventive method and article enables owners of existing ozone-generating machines to use those machine in a new way, namely to concentrate the ozone generated by their ozone-generating machines into specific confined volumes such as garment bags and athletic bags, to benefit from the application of ozone to specific articles such as men's suits and shoes.

### BACKGROUND OF THE INVENTION

1. Field of Invention

This invention pertains to the art of methods and articles and apparatuses for concentrating and transporting ozone or other desirable gases at desired locations, usually remote from the source of such ozone or other desirable gases.

2. Description of the Related Art

Ozone generators are known. Typical ozone generators are available from companies such as Alpine Industries.

### SUMMARY OF THE INVENTION

The invention concerns a method of concentrating ozone to a desired location. The method comprises the steps of generating ozone by an ozone generating machine, said ozone generating machine having an ozone generating machine outlet for the associated ozone. Further, then concentrating the ozone at a desired location. The desired location is often remote from the ozone generating machine. According to another aspect of the invention, an article for concentrating ozone generated by an ozone generating machine includes an ozone generating machine and a channeler.

The channeler directs ozone generated by the ozone generating machine to a desired location. The desired location is remote from the ozone generating machine. The channeler extends from an ozone generating machine outlet to the desired location. The ozone has a first concentration at the ozone generating machine outlet and a second concentration at the desired location.

The channeler further comprises attaching means for attaching the channeler to the ozone-generating machine. The channeler includes a first end and a second end. The first end has a first cross-sectional area and the second end has a second cross-sectional area. The first cross-sectional area is greater than the second cross-sectional area, so that ozone is concentrated as the ozone passes from the first end to the second end of the channeler. The channeler also includes an outlet which is capable of receiving a hose. The hose has first and second ends with the first end of the hose being operatively associated with the outlet of the ozone generating machine and the second end of the hose being operatively associated with a mattress bag. Instead of the mattress bag, other concentrating implements can be used, some concentrating implements include the mattress bag, a garment bag, a dryer vent, and a remote concentrator. The remote concentrator has a first side and a second side. The first side cooperates with the second end of the hose and the second side of the remote concentrator is selectively placeable over a generally planar location to be treated.

Still other benefits and advantages of the invention will become apparent to those skilled in the art to which it pertains upon a reading and understanding of the following detailed specification.

### BRIEF DESCRIPTION OF THE DRAWINGS

The invention may take physical form in certain parts and arrangement of parts, a preferred embodiment of which will be described in detail in this specification and illustrated in the accompanying drawings which form a part hereof and herein:

FIG. 1 is a perspective view of an ozone-generating machine according to the invention;

FIG. 2 is a perspective view of a remote concentrator according to the invention;

FIG. 3 is a bottom view of a concentrating implement, specifically a brush;

FIG. 4 is a side view of the concentrating implement, in this case a brush, shown in FIG. 3; and,

FIG. 5 is a perspective view of another concentrating implement, in this case a garment bag.

### DESCRIPTION OF THE PREFERRED EMBODIMENT

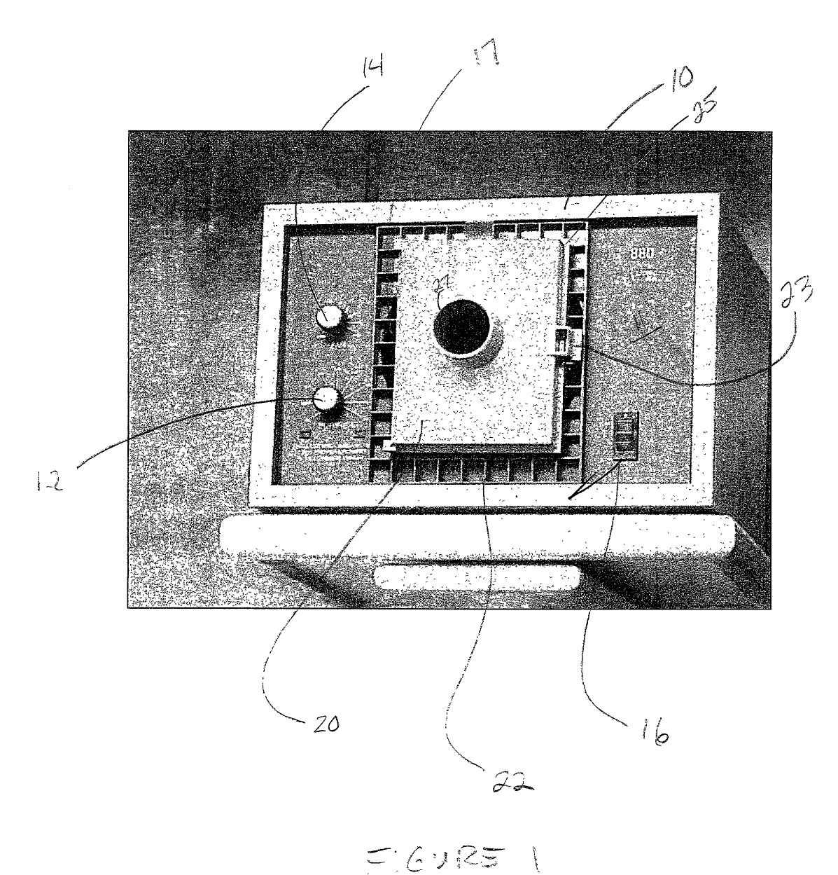

Referring now to the drawings wherein the showings are for purposes of illustrating a preferred embodiment of the invention only and not for purposes of limiting the same, FIG. 1 shows an oxygen-generating machine 10. The oxygen-generating machine 10 can be of any form or design if design and manufactured according to sound engineering knowledge. The oxygen-generating machine 10 is typically cubicle, including a front side 11. In the embodiment illustrated in FIG. 1 the front side 11 of the oxygen-generating machine 10 includes a knob 12 which controls the amount of ozone generated by the machine. In addition, a knob 14 controls the speed of a fan (not shown) which is included inside the oxygen-generating machine. By manipulating the knob 12 and the knob 14, varying amounts of ozone can be generated and pushed out of the oxygen-generating machine. In addition to knobs 12 and 14, the front side 11 of the oxygen-generating machine 10 also includes an on-off switch 16.

Continuing reference to FIG. 1, all oxygen-generating machines, including the embodiment shown in FIG. 1, have some sort of an outlet for the ozone. In the present case, the outlet 17 of the oxygen-generating machine 10 is on the front side 11. In the preferred embodiment oxygen-generating machine 10, the outlet 17 is covered by grill 22. In one embodiment of the invention, a channeler 20 is attached to the grill 22 the attaching means 23. In another embodiment of the invention, the entire grill 22 is removed and replaced by the channeler 20.

With continuing reference to FIG. 1, the channeler includes a first end and a second end. The first end 25 of the channeler lies next to the outlet 17 of the oxygen-generating machine 10. The second end 27 of the channeler 20 is spaced a distance away from the outlet 17 of the oxygen-generating machine 10. In one embodiment, the second end 27 of the channeler functions as an outlet for the ozone. In such cases, the channeler 20 might be fairly large with a significance distance between the second end 27 and the first end 25. However, in the preferred embodiments illustrated herein, the second end 27 of the channeler 20 is configured to receive a hose 30.

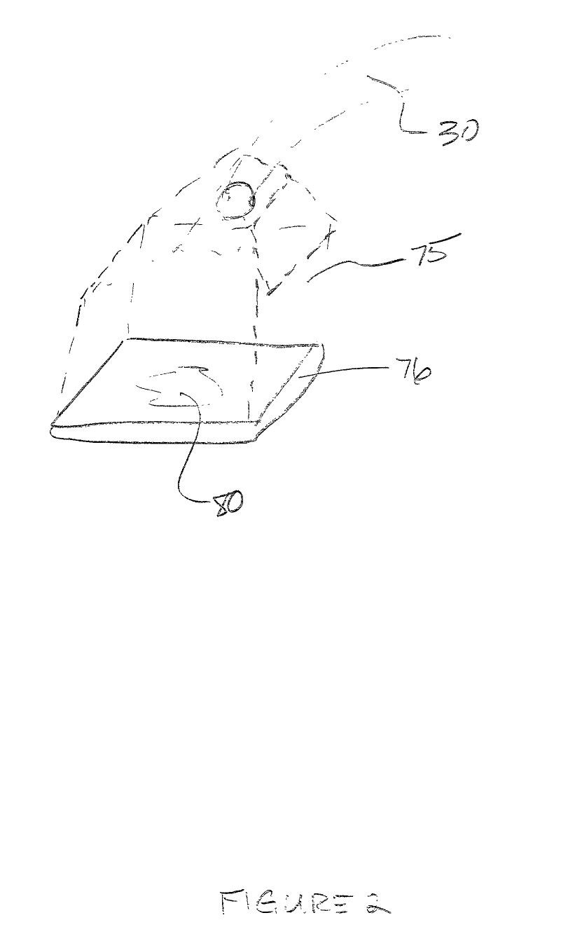

With reference to FIG. 2, the hose 30 has a first end 31 and a second end 32. The first end of the hose 30 is operatively associated with the outlet 17 of the ozone-generating machine. In another, preferred embodiment, the first end 31 of the hose is operatively associated with the second end 27, or outlet, of the channeler 20. The second end 32 of the hose 30 is operatively associated with any one of a variety of concentrating implements. Among the implements within the scope of the invention are shoe bags, athletic bags, luggage, a mattress bag, a garment bag, a dryer vent, and a remote concentrator. Many or all of these items are well known and are not fully described herein. However, for illustration sake, a garment bag 40 is illustrated. The second end 32 of the hose 30 is affixed in a operative way to a garment bag 40. The garment bag also includes an opening 42 most likely opened and closed by a zipper 43. In operation, a user would hang one or more articles of clothing, such as a man's suit, into the garment bag 40. As such time, the hose 30 would be attached and the zipper 43 would be shut. Then the ozone-generating machine 10 would be turned on and a desired amount of ozone would be generated by the machine 10 and delivered through the hose 30 to the interior of the garment bag 40. The provision of the ozone would provide all of the benefits associated and believed to flow from the use of the product.

The use of the ozone-concentrating method and articles disclosed herein is essentially the same regardless of the concentrating implement used. For example, in FIG. 5, the garment bag 40 could be replaced with a shoe bag, an athletic bag, luggage, a dryer vent, or a mattress bag. In each case, the second end 32 of the hose 30 would be provided in an operative sense to the interior of the article in question, such as a mattress bag. Inside the article, such as a mattress bag, would be articles to be treated with ozone. In the case of a mattress bag, it would be a mattress for a bed. Then the oxygen concentrating machine 10 would be turned on until a desired amount of ozone is delivered to the article in question.

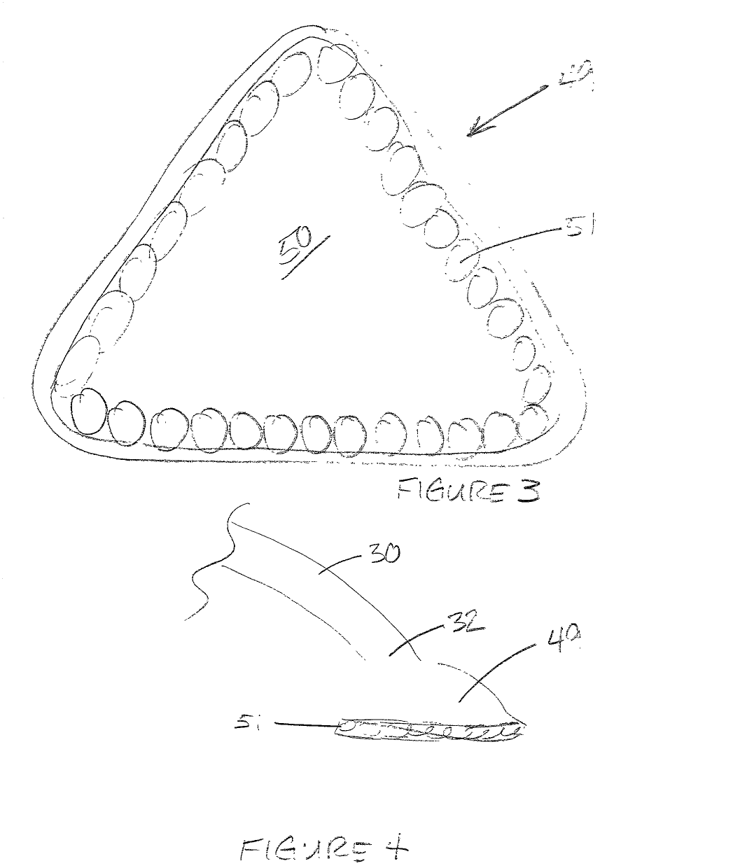

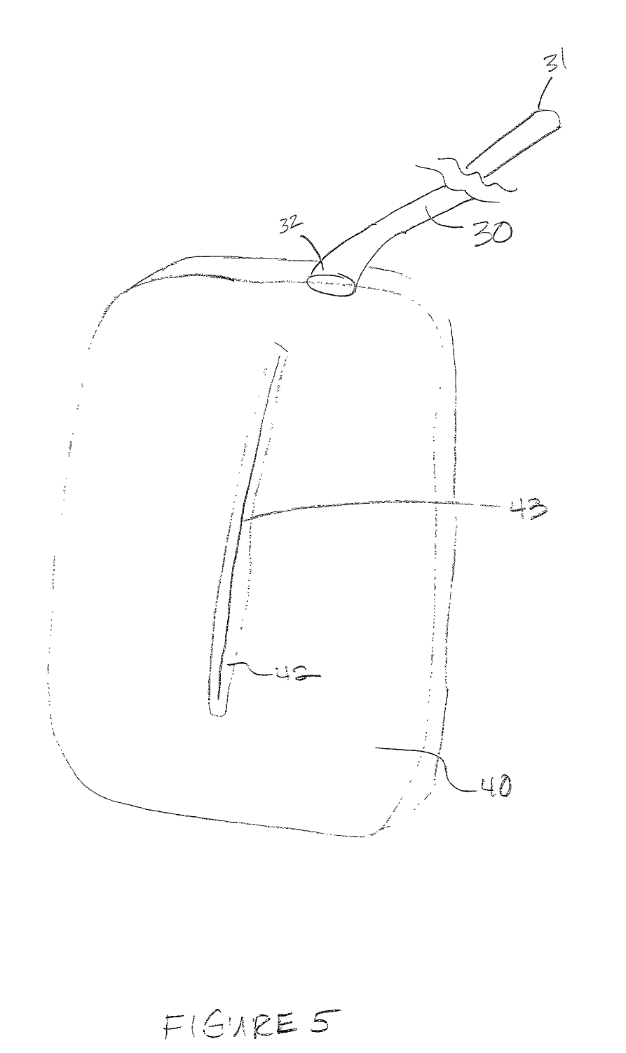

With reference to FIGS. 3 and 4, a specific type of concentrating implement is illustrated. In this case, the concentrating implement is a brush 49. In this case, this concentrating implement features an underside 50 as illustrated in FIG. 3. Around the periphery of the underside are bristles 51. In one embodiment, the bristles 51 would be somehow powered in a linear or rotary manner so that agitation could occur. Such devices are commonly known in the art, but not with the provision of ozone. As illustrated in FIG. 4, such a product might provide vacuum, ozone, in agitation all at one time.

With reference to FIG. 2, a remote concentrator 75 is illustrated. The footprint of the remote concentrator 75 can be of any configuration, such as a rectangle 76 as illustrated in FIG. 2. The manner of using this remote concentrator would be to place it over a spot 80. This spot could be in a variety of locations, such as on the floor or on the cushion of a sofa or chair. In any event, this remote concentrator 75 would seem to work most efficiently when it was set upon a planar surface such as a floor. The remote concentrator 75 would appear to be ideal for pet odors and the like.

With continuing reference to FIGS. 3 and 4, is within the scope of the invention that the provision of ozone generated by a ozone-generating machine 10 to a desired remote location via a remote concentrator and a hose includes the use of this process and article with more conventional floor care devices such as a rug shampooer. For example, in FIGS. 3 and 4, the concentrating implement shown could be used to shampoo a rug and, if provided with air powered agitation, sweep or fresh in a rug. The essence of the invention is the provision of an article and method for transporting ozone generated at one location to a second, remote location and perhaps concentrating it further. One of the most important parts and aspects of the invention is that the ozone-concentrating devices including the hose 30, the channeler 20, and the various concentrating implements, are all selectively removable from the oxygen-generating machine 10. This feature allows all of these implements to be purchased and applied retroactively to existing ozone-generating machine 10. In addition, in the case of a new ozone-generating machine 10, the ozone-generating machine can be allowed to run in its normal operation in a room but, in addition to that normal operation, the inventive process and articles herein described allow the ozone-generating machine 10 to treat certain specific items, such as a mattress or a man's suit, or certain spots on the rug, such as where a pet has soiled the carpet.

The invention has been described with reference to preferred embodiment. Obviously, modifications and alterations will occur to others upon a reading and understanding of this specification. It is intended to include all such modifications and alternations in so far as they come within the scope of the appended claims or the equivalence thereof.

Having thus described the invention, it is now claimed:

What is claimed is:

1. A method of concentrating ozone to a desired location, the method comprising the steps of:
generating ozone by an ozone-generating machine, said ozone-generating machine having an ozone-generating machine outlet for the associated ozone; and,
concentrating said ozone at a desired location.

2. The method of claim 1 wherein the desired location is remote from the ozone-generating machine.

3. The method of claim 1 wherein said step of concentrating said ozone includes a step of:
Transporting said ozone from said ozone-generating machine outlet via a channeler.

4. The method of claim 2 wherein said ozone has a first concentration at said ozone-generating machine outlet, and said ozone has a second concentration at the desired location.

5. The method of claim 4 wherein said first concentration of ozone is greater than said second concentration of ozone.

6. An article for concentrating ozone generated by an associated ozone-generating machine, said article comprising:
A channeler, said channeler directing ozone generated by the associated ozone-generating machine to a desired location.

7. The article of claim 6 wherein the desired location is remote from the ozone-generating machine, said channeler extending from an ozone-generating machine outlet to the desired location.

8. The article of claim 6 wherein said ozone has a first concentration at the ozone-generating machine outlet, and said ozone has a second concentration at the desired location.

9. The article of claim 6 wherein said channeler further comprises:
Attaching means for attaching said channeler to said ozone-generating machine.

10. The article of claim 6 wherein said channeler includes a first end and a second end, said first end having a first cross-sectional area and said second end having a second cross-sectional area, said first cross-sectional area being greater than said second cross-sectional area, so that ozone is concentrated as the ozone passes from said first end to said second end of the channeler.

11. The article of claim 6 wherein said channeler further comprises:
A channeler outlet; said channeler outlet capable of receiving a hose.

12. The article of claim 11 further comprising:
A hose; said hose having first and second ends; said hose extending from said channeler outlet to said desired location.

13. The article of claim 12 wherein said first end of said hose is operatively associated with said outlet of said ozone-generating machine and said second end of said hose is operatively associated with a mattress bag.

14. The article of claim 12 wherein said first end of said hose is operatively associated with said outlet of said ozone-generating machine and said second end of said hose is operatively associated with a garmet bag.

15. The article of claim 12 wherein said first end of said hose is operatively associated with said outlet of said ozone-generating machine and said second end of said hose is operatively associated with a dryer vent.

16. The article of claim 12 wherein said first end of said hose is operatively associated with said outlet of said ozone-generating machine and said second end of said hose is operatively associated with a bag, said bag having a reusable and adjustable opening so that associated articles can be placed within the associated bag.

17. The article of claim 12 wherein said first end of said hose is operatively associated with said outlet of said ozone-generating machine and said second end of said hose is operatively associated with a remote concentrator.

18. The article of claim 17 wherein said remote concentrator has a first side and a second side, said first side cooperating with said second end of said hose, said second side of said remote concentrator being selectively placeable over a generally planar location to be treated.

19. The article of claim 12 wherein said first end of said hose is operatively associated with said outlet of said ozone-generating machine and said second end of said hose is operatively associated with a shoe bag.

20. An article for concentrating ozone generated by an ozone-generating machine, said article comprising:
An ozone generating machine;
A channeler, said channeler directing ozone generated by said ozone-generating machine to a desired location; the desired location is remote from the ozone-generating machine, said channeler extending from an ozone-generating machine outlet to the desired location, said ozone having a first concentration at the ozone-generating machine outlet, and a second concentration at the desired location; said channeler further comprising:
attaching means for attaching said channeler to said ozone-generating machine;
a first end and a second end, said first end having a first cross-sectional area and said second end having a second cross-sectional area, said first cross-sectional area being greater than said second cross-sectional area, so that ozone is concentrated as the ozone passes from said first end to said second end of the channeler; and,
A channeler outlet; said channeler outlet capable of receiving said hose.
a hose; said hose having first and second ends; said hose extending from said channeler outlet to said desired location; said first end of said hose being operatively associated with said outlet of said ozone-generating machine and said second end of said hose is operatively associated with a concentrating implement.
